(19) United States
(12) Patent Application Publication
(10) Pub. No.: US 2025/0213678 A1
(43) Pub. Date: Jul. 3, 2025

(54) VACCINE DEVELOPMENT PLATFORM USING PLANT-SECRETED NANO-VESICLES FOR DELIVERY OF RECOMBINANT PROTEIN AND mRNA

(71) Applicant: Andong National University Industry-Academic Cooperation Foundation, Andong-si (KR)

(72) Inventor: Young-Eun CHO, Andong-si (KR)

(73) Assignee: Andong National University Industry-Academic Cooperation Foundation, Andong-si (KR)

(21) Appl. No.: 18/988,213

(22) Filed: Dec. 19, 2024

(30) Foreign Application Priority Data
Dec. 28, 2023 (KR) 10-2023-0194044

(51) Int. Cl. A61K 39/29; A61K 9/00; A61K 9/51; A61K 39/00; A61P 31/20; B82Y 5/00

(52) U.S. Cl. CPC A61K 39/292; A61K 9/0053; A61K 9/5176; A61P 31/20; A61K 2039/55555; A61K 2039/575; B82Y 5/00

(57) ABSTRACT

Proposed is a new vaccine development platform using plant-secreted nano-vesicles for delivery of recombinant proteins and mRNA. It was confirmed that the delivery efficiency of the hepatitis B virus surface antigen (HBsAg) (drug) was higher when loaded onto nano-vesicles isolated from grapefruit or mandarin orange, compared to administering the antigen alone. Thereby, the nano-vesicles can be utilized as a composition for drug delivery.

### STATEMENT REGARDING GOVERNMENT SPONSORED RESEARCH

This invention was supported by Andong of Korea. Research Project name: “Andong type job”; Research Subject name: “Development of immune enhancers and vaccine adjuvants using plant-derived nano-vesicles”; Research Subject Number: 2024-0295

### CROSS REFERENCE TO RELATED APPLICATION

The present application claims priority to Korean n Patent Application No. 10-2023-0194044, filed Dec. 28, 2023, the entire contents of which is incorporated herein for all purposes by this reference.

### BACKGROUND OF THE DISCLOSURE

### 1. Field of the Disclosure

The present disclosure relates to a new vaccine development platform using plant-secreted nano-vesicles for delivery of recombinant proteins and mRNA.

### 2. Description of the Related Art

Vaccination has been considered the most efficient method to control various infectious diseases caused by viruses and pathogenic microorganisms from a cost perspective. As seen with the current COVID-19 pandemic, the absence of an effective vaccine against newly emerged viruses could pose a serious threat to public health worldwide. Recombinant protein vaccines, mRNA vaccines, and viral vector vaccines have been designed as next-generation vaccine platforms in addition to classic types of vaccines such as inactivated vaccines and live attenuated vaccines. In addition to the vaccine platforms, the development of new vaccine delivery tools is also important to improve vaccine efficacy by enhancing antigen delivery to immune cells.

Meanwhile, extracellular vesicles (hereinafter referred to as EVs) are small vesicles that produce various types of cells. The extracellular vesicles have emerged as a new delivery system for small interfering RNA (siRNA) or pharmaceutically active substances, and even vaccine antigens. Plants release nano-vesicles similar to exosomes, which play a role in communication between plant cells. It has been reported that these nano-vesicles induce various biological activities by delivering miRNA, mRNA, and proteins. In addition, while mammalian-derived nano-vesicles are difficult to mass-produce, plant-derived exosome-like nano-vesicles (PENVs) can be easily isolated and purified in large quantities, and the plant-derived exosome-like nano-vesicles (PENVs) are non-toxic and stable because the plant-derived exosome-like nano-vesicles (PENVs) are obtained from edible plants. Despite the many advantages of plant-derived exosome-like nano-vesicles, research on the use thereof as a new delivery tool is still insufficient.

### RELATED ART DOCUMENT

### Patent Document

(Patent document 1) 1. Korean Patent No. KR 10-2065819 (registered on 2020.01.07.)

### SUMMARY OF THE DISCLOSURE

The present disclosure is to provide a composition for drug delivery including plant-derived nano-vesicles as an active ingredient.

To achieve the objective, the present disclosure provides a drug delivery composition including nano-vesicles derived from grapefruit (Citrus× paradisi) (hereinafter referred to as grapefruit derived nano-vesicles) or nano-vesicles derived from mandarin orange (Citrus reticulata) (hereinafter referred to as mandarin orange derived nano-vesicles) as an active ingredient.

In addition, the present disclosure provides a pharmaceutical composition for preventing or treating hepatitis B, the pharmaceutical composition including nano-vesicles, which are loaded with a hepatitis B treatment drug, as an active ingredient.

In addition, the present disclosure provides a health functional food composition for preventing or alleviating hepatitis B, the health functional food composition including nano-vesicles, which are loaded with a hepatitis B treatment drug, as an active ingredient.

According to the present disclosure, it was confirmed that the delivery efficiency of the hepatitis B virus surface antigen (HBsAg) (drug) was higher when loaded onto nano-vesicles isolated from grapefruit or mandarin orange, compared to administering the antigen alone. Thereby, the nano-vesicles can be utilized as a composition for drug delivery.

### BRIEF DESCRIPTION OF THE DRAWINGS

FIGS. 1A and 1B show the results of analyzing the size and shape of grapefruit derived nano-vesicles (hereinafter referred to as GNVs) and mandarin orange derived nano-vesicles (hereinafter referred to as MNVs) (hereinafter, the GNVs and MNVs are referred to as samples);

FIGS. 2A and 2B show the results of analyzing the cytotoxicity of the samples in kidney cells, and data are expressed as mean+error (SEM) for three independent experiments;

FIGS. 3A and 3B show the results of analyzing whether samples labeled with 1,1′-dioctadecyl-3, 3,3′, 3′-tetramethylindotricarbocyanine iodide (DiRDiR, DiIC18 (7)) are well absorbed in kidney cells/organs;

FIGS. 4A to 4F show the results of analyzing the drug delivery capacity of the samples; and

FIGS. 5A and 5B show the results of analyzing the effect of the samples on immunogenicity in drug delivery.

### DESCRIPTION OF THE PREFERRED EMBODIMENTS

Hereinafter, the present disclosure will be described in more detail.

The present disclosure provides a drug delivery composition including nano-vesicles derived from grapefruit (Citrus× paradisi) (hereinafter referred to as grapefruit derived nano-vesicles) or nano-vesicles derived from mandarin orange (Citrus reticulata) (hereinafter referred to as mandarin orange derived nano-vesicles) as an active ingredient.

The grapefruit derived nano-vesicles may have an average particle diameter in a range of 30 to 60 nm. The mandarin orange derived nano-vesicles may have an average particle diameter in a range of 100 to 300 nm.

The nano-vesicles may improve delivery efficiency of a drug into cells. The drug may include hepatitis B virus surface antigen (HBsAg) or influenza virus antigen, but the type is not limited thereto.

In addition, the present disclosure provides a pharmaceutical composition for preventing or treating hepatitis B, the pharmaceutical composition including nano-vesicles, which are loaded with a hepatitis B treatment drug, as an active ingredient.

The hepatitis B treatment drug may include hepatitis B virus surface antigen (HBsAg).

The pharmaceutical composition of the present disclosure is formulated using a pharmaceutically acceptable carrier by a method, which may be easily implemented by those skilled in the art to which the present disclosure pertains. Thereby, the pharmaceutical composition of the present disclosure may be prepared in unit dosage form or by placing the pharmaceutical composition in a multi-dose container.

The pharmaceutically acceptable carrier may be one commonly used in preparation. The pharmaceutically acceptable carrier may include lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil but is not limited thereto. In addition to the ingredients, the pharmaceutical composition of the present disclosure may further include lubricants, wetting agents, sweeteners, flavoring agents, emulsifiers, suspending agents, and preservatives.

In the present disclosure, the content of additives included in the pharmaceutical composition is not particularly limited and may be appropriately adjusted within the content range used in conventional formulations.

The pharmaceutical composition may be formulated in the form of one or more external skin preparations selected from the group consisting of injectable dosage forms (such as aqueous solutions, suspensions, and emulsions), pills, capsules, granules, tablets, creams, gels, patches, sprays, ointments, warning agents, lotions, liniment agents, pasta agents, and cataplasma agents but is not limited thereto.

The pharmaceutical composition of the present disclosure may further include a pharmaceutically acceptable carrier and diluent for formulation. The pharmaceutically acceptable carrier and diluent may include excipients such as starches, sugars, and mannitol, fillers and extenders such as calcium phosphate, cellulose derivatives such as carboxymethylcellulose and hydroxypropylcellulose, binding agents such as gelatin, alginate, and polyvinyl pyrrolidone, lubricants such as talc, calcium stearate, hydrogenated castor oil, and polyethylene glycol, disintegrants such as povidone and crospovidone, surfactants such as polysorbate, cetyl alcohol, and glycerol but are not limited thereto. The pharmaceutically acceptable carrier and diluent may be biologically and physiologically friendly to the subject. The diluent may include saline, aqueous buffers, solvents, and/or dispersion media but is not limited thereto.

The pharmaceutical composition of the present disclosure may be administered orally or parenterally (for example, intravenously, subcutaneously, intraperitoneally, or topically) depending on the preferred method. In the case of oral administration, the pharmaceutical composition of the present disclosure may be formulated as tablets, troches, lozenges, aqueous suspensions, oily suspensions, prepared powders, granules, emulsions, hard capsules, soft capsules, syrups, or elixirs. In the case of parenteral administration, the pharmaceutical composition of the present disclosure may be formulated as an injection solution, suppository, powder for respiratory inhalation, aerosol for spray, ointment, powder for application, oil, or cream.

The dosage of the pharmaceutical composition of the present disclosure may vary depending on the patient's condition, weight, age, gender, health, dietary constitution specificity, nature of the preparation, degree of disease, administration time of the composition, administration method, administration period or interval, excretion rate, and drug form. The dosage may be appropriately selected by those skilled in the art. The dosage may be, for example, in the range of about 0.1 to 10,000 mg/kg but is not limited thereto, and the pharmaceutical composition of the present disclosure be may administered once or in divided doses several times a day. The pharmaceutical composition may be administered orally or parenterally (for example, intravenously, subcutaneously, intraperitoneally, or topically) depending on the preferred method.

The pharmaceutically effective amount and effective dosage of the pharmaceutical composition of the present disclosure may vary depending on the formulation method, administration method, administration time, and administration route of the pharmaceutical composition. Those skilled in the art may easily determine and prescribe an effective dosage for the desired treatment. The pharmaceutical composition of the present disclosure may be administered once a day or may be administered in several divided doses.

In addition, the present disclosure provides a health functional food composition for preventing or alleviating hepatitis B, the health functional food composition including nano-vesicles, which are loaded with a hepatitis B treatment drug, as an active ingredient.

The hepatitis B treatment drug may include hepatitis B virus surface antigen (HBsAg).

The present disclosure may be generally used as commonly used foods.

The food composition of the present disclosure may be used as a health functional food. The term “health functional food” refers to food prepared and processed by using raw materials or ingredients with functional properties beneficial to the human body in accordance with the Health Functional Food Act. The term “functionality” refers to consumption-driven beneficial effects for health, such as regulating nutrients or exerting physiological actions on the structure and function of the human body.

The health functional food composition may contain common food additives. Unless otherwise specified, suitability as a “food additive” is determined on the basis of the specifications and standards for relevant items following the general rules and general test methods outlined in the Food Additives Code approved by the Ministry of Food and Drug Safety.

The items listed in the “Food Additives Code” may include, for example, chemical compounds such as ketones, glycine, potassium citrate, nicotinic acid, and cinnamic acid, natural additives such as dark pigment, licorice extract, crystalline cellulose, high-quality pigment, and guar gum, mixed preparations such as L-glutamate sodium preparation, noodle additive alkaline preparation, preservative preparation, and tar coloring preparation.

The food composition of the present disclosure may be prepared and processed in the form of tablets, capsules, powders, granules, liquids, and pills. For example, among health functional foods in the form of capsules, hard capsules may be prepared by mixing the composition of the present disclosure with additives such as excipients and filling the mixture in a conventional hard capsule, and soft capsules may be prepared by mixing the composition of the present disclosure with additives such as excipients and filling the mixture in a capsule base such as gelatin. The soft capsules may contain glycerin or sorbitol, colorants, and plasticizers such as preservatives, if necessary.

Definitions of terms such as excipients, binders, disintegrants, lubricants, coagulants, and flavoring agents are described in literature known in the art and include those with the same or similar functions. There is no particular limitation on the type of food, and the food includes all health functional foods in the conventional sense. In the present disclosure, the term “prevention” refers to all actions that suppress or delay diseases by administering the composition of the present disclosure.

In the present disclosure, the term “treatment” refers to any action that alleviates or beneficially changes the symptoms of diseases by administering the composition of the present disclosure.

In the present disclosure, the term “improvement” refers to any state caused by all actions that alleviate bad conditions caused by the diseases by administering the composition of the present disclosure. Hereinafter, the present disclosure will be described in detail through examples to aid understanding. However, the following examples only illustrate the content of the present disclosure, and the scope of the present disclosure is not limited to the following examples. Examples of the present disclosure are provided to more completely explain the present disclosure to those skilled in the art.

### [Experimental Example 1] Experiment Preparation

### 1-1. Sample

Grapefruit (Citrus×paradisi) and Mandarin orange (Citrus reticulata) were collected in Israel and Korea (Jeju Island). The grapefruit and mandarin orange were washed with distilled water and peeled. Afterward, the grapefruit and mandarin orange were mixed with cold phosphate-buffered saline (PBS), to obtain fruit juice. Each fruit juice was sequentially centrifuged. Each obtained pellet was resuspended in the PBS to prepare grapefruit derived nano-vesicles (GNVs) and mandarin orange derived nano-vesicles (MNVs) used as samples of the present disclosure. Aliquots of the samples were stored at a temperature of −80° C. until use. The protein concentration of GNVs and MNVs was measured, respectively, by a BCA analysis.

### 1-2. Cell Model

Madin-Darby Canine Kidney (MDCK) cells and Vero cells (cells derived from renal epithelial cells of African green monkeys) were purchased from ATCC and used (MDCK cells; CCL-34 and Vero cells; CCL-81). The cells were seeded in Dulbecco's Modified Eagle Medium (DMEM, Hyclone Laboratories, Inc., Logan, UT, USA) containing 10% fetal bovine serum (FBS) and 1% penicillin, respectively, and incubated under conditions of 5% CO and 37° C.

### 1-3. Animal Model

Six-week-old male BALB/c mice were purchased from Koatech (Pyeongtaek, Korea). The mice were allowed to freely consume food and water under a 12-hour light/dark cycle.

### [Experimental Example 2] Nano-Particle Tracking and Transmission Electron Microscope Analysis

A nano-particle tracking analysis (NTA, Nanosight NS300, Malvern Instruments, Malvern, UK) and negative staining transmission electron microscope (TEM) analysis were performed on the size and shape of the samples. Sample images were visualized using the TEM. The sample images were obtained by staining the samples with 0.75% uranyl formate and coating the samples on a glow-discharged copper grid. The samples were examined using a JEM-1010 transmission electron microscope (JEOL, Tokyo, Japan) at an acceleration voltage of 100 kV.

### [Experimental Example 3] MTT Assay

An MTT assay was performed to confirm the cytotoxicity of the samples in kidney cells. The MDCK and Vero cells were dispensed into 96-well plates and incubated under Experimental Example 1-2 conditions. After 24 hours, the cells were treated with the samples at different concentrations and cultured for 24 hours. Afterward, 3-[4, 5-dimethylthiazol-2-y]-2, 5-diphenyltetrazolium bromide (MTT, Sigma, St. Louis, MO, USA) was used to analyze cytotoxicity. Formazan was dissolved in dimethyl sulfoxide (DMSO), and then absorbance was measured at a wavelength of 570 nm using a microplate ELISA reader (Infinite m200pro; Tecan, Grödig, Austria).

### [Experimental Example 4] Sample-DiR Labeling and Long-Term Imaging

Sample labeling was performed in accordance with the manufacturer's instructions (ThermoFisher, D12731) by using 1,1′-dioctadecyl-3, 3, 3′, 3′-tetramethylindotricarbocyanine iodide (DiR, DiIC18 (7)). Briefly, 50 μg/mL of samples was resuspended in 10 μL of 1 m MDiR stock solution and incubated for 30 minutes at room temperature. DIR-labeled samples were separated into pellets using an ultracentrifuge (100,000 g×1). The pellets were resuspended in 1 mL PBS, and each supernatant was discarded. Afterward, the MDCK and Vero cells were cultured with 1 μM DiR-labeled samples for 4, 8, and 12 hours, respectively. For nuclear staining, DAPI (Sigma Aldrich, USA) was mounted under a glass coverslip, and each slide was visualized through immunofluorescence microscopy.

For long-term imaging, the DiR-labeled samples were orally administered to the mice of Experimental Example 1-3 (1 mg protein, 1×10¹²/g body weight), and the organs of the mice were collected after 4, 8, 12, and 24 hours, respectively. The collected organs were excised and imaged with an Odyssey scanner (LI-COR, USA).

### [Experimental Example 5] Loading Protein onto Samples

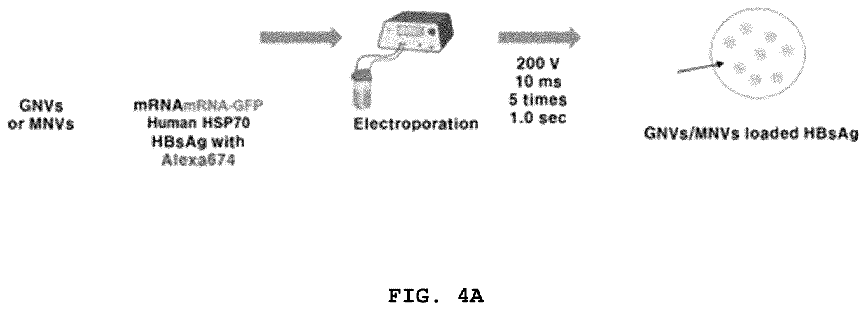

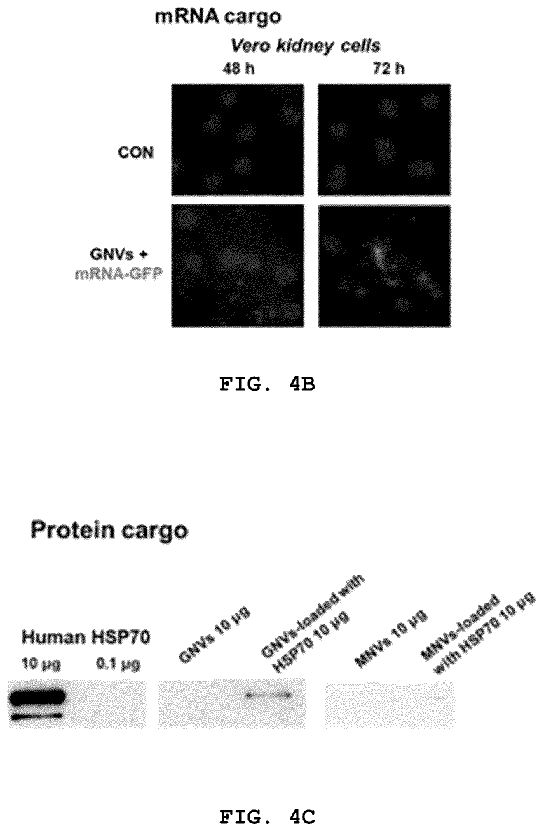

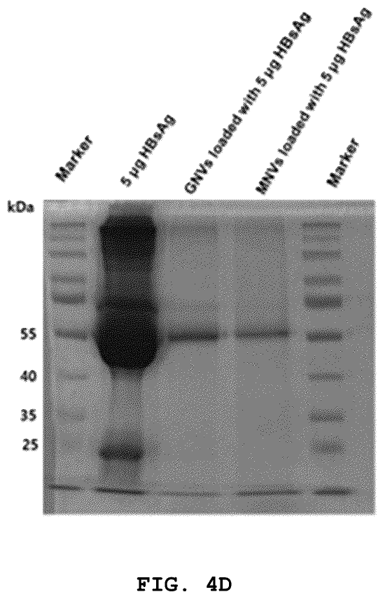

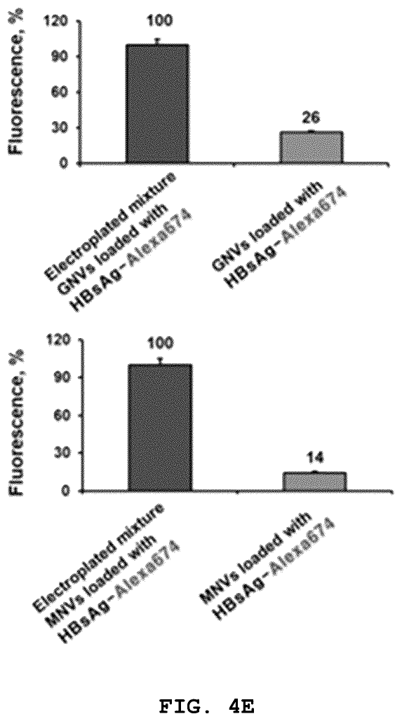

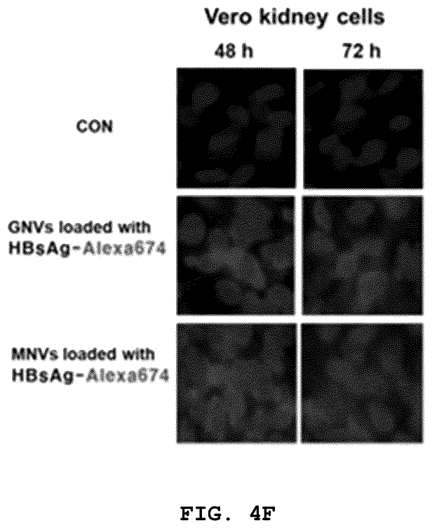

As shown in FIG. 4A, mRNA-GFP (green fluorescent protein), human heat shock protein 70 (HSP70) protein, and hepatitis B virus surface antigen (hereinafter referred to as HBsAg) recombinant protein were loaded onto the samples through electrophoresis. Each loaded sample was diluted with PBS (nanovesicle: PBS=1:9 weight ratio). The mRNA-GFP (1 ng), human HSP70 protein (10 μg), and HBsAg recombinant protein (5 μg) were added to the buffer solution. The mRNA-GFP, human HSP70 protein, and HBsAg recombinant protein were transferred to an ice-cold 0.4 cm Gene Pulser/MicroPulser cuvette (Electroporation Cuvette). Afterward, the electroporation cuvette was inserted into the Gene Pulser Xcell Total System and subjected to electrophoresis (voltage: 200V, voltage difference: 10 ms, number of iterations: 5 and voltage time: 1 second).

### [Experimental Example 6] Western Blotting Analysis

The same amount of proteins as the human HSP70 protein loaded onto the samples were separated by SDS-PAGE under reducing conditions. The proteins were transferred to a polyvinylidene fluoride (PVDF) membrane at a voltage of 120 V for 30 minutes. Blocking was performed for 2 hours at room temperature using 5% skim milk dissolved in PBST (PBS containing 0.1% Tween-20), followed by overnight incubation at a temperature of 4° C. with a primary antibody against HSP (abcam). Then, another incubation was performed with a secondary antibody conjugated with horseradish peroxidase. Next, western blotting results were visualized using enhanced chemiluminescence (Super Pico Detection Reagent, Pierce: Rockford, IL, USA). Quantification was performed using ChemiDoc Gel Quantification System (Bio-Rad: Hercules, CA, USA).

### [Experimental Example 7] Vaccination in Animal Model

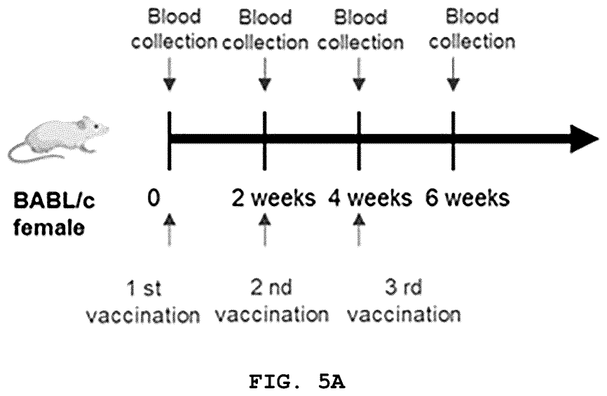

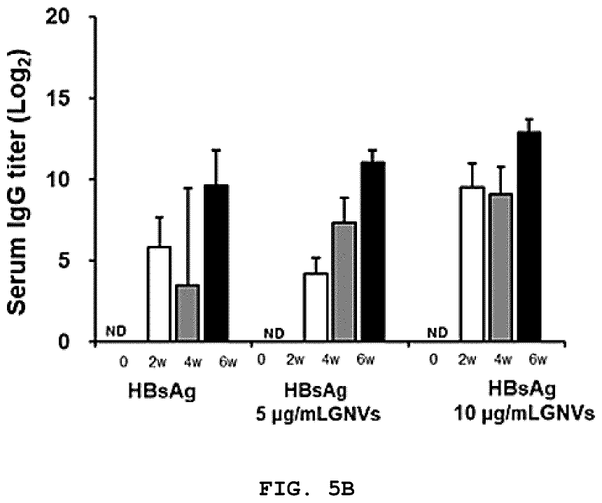

Six-week-old male BALB/c mice (Koatech, Pyeongtaek, Korea) were allowed to consume food and water freely under a 12-hour light/dark cycle. Then, as shown in FIG. 5A, the HBsAg recombinant protein loaded onto GNVs was inoculated into the mice by oral administration twice a week. A control group (HBsAg) was HBsAg recombinant protein (10 μg/100 μL), which was not loaded onto nano-vesicles (a conventional vaccine administration method), and the control group was inoculated into mice by intramuscular injection a total of twice a week. Next, the mice were anesthetized, and their blood was collected through orbital blood collection.

### [Experimental Example 8] ELISA Assay

To determine the extent to which HBsAg was loaded onto GNVs and MNVs in mouse serum, an ELISA analysis was performed. The HBsAg protein loaded onto the samples was coated onto each well of a 96-well plate. After blocking, the plates were washed and incubated at room temperature for 1 hour with a five-fold serially diluted serum, followed by incubation with an HRP-conjugated anti-mouse IgG antibody at room temperature for 1 hour. After washing, the plates were incubated with tetramethylbenzidine (TMB) solution at room temperature for 5 minutes. The reactions were stopped by adding 1 N HCl solution, and absorbance was measured at a wavelength of 450 nm using an ELISA reader.

### [Experimental Example 9] Statistical Assay

Data were analyzed using SPSS Statistics (ver. 27.0, SPSS Inc., Chicago, IL, USA). Statistical significance was set at the mean difference of p<0.05 using Tukey's HSD post-hoc analysis and one-way analysis of variance (ANOVA).

### [Example 1] Analysis of Sample Size and Shape

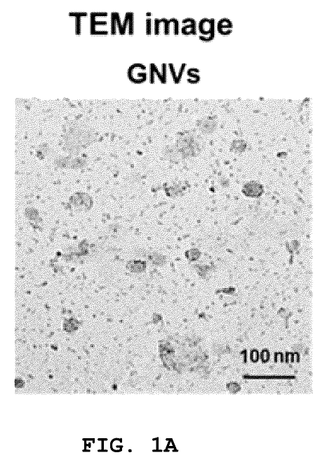

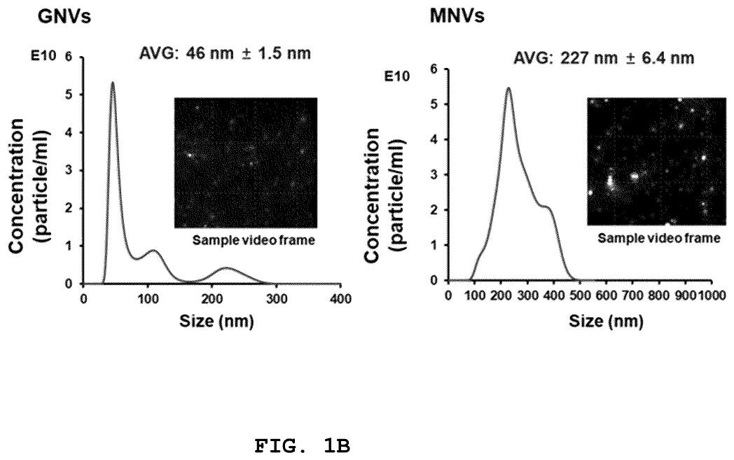

According to Experimental Example 2 above, as a result of analyzing the size and shape of the samples, as shown in FIGS. 1A and 1B, it was confirmed that an average particle diameter (size) of GNVs and MNVs was 46±1.5 nm and 227±6.4 nm at 106 particles/mL, respectively, and the GNVs and MNVs had a round shape with a size range of <100 nm (FIGS. 1A and 1B). From the results, it was confirmed that GNVs and MNVs were successfully separated as nano-vesicles.

### [Example 2] Cytotoxicity Analysis

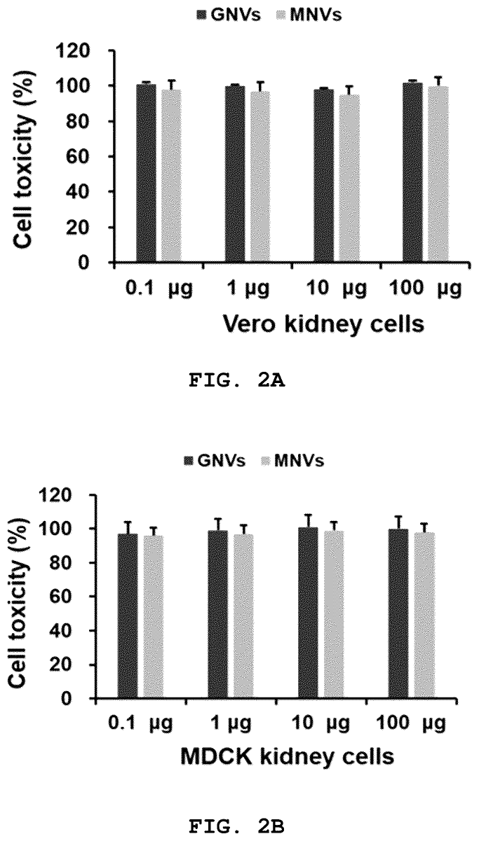

According to Experimental Example 3, as a result of analyzing the cytotoxicity of the samples in kidney cells, as shown in FIGS. 2A and 2B, there was no significant change in cell viability in the sample treatment group (FIGS. 2A and 2B). From the result, it was confirmed that GNVs and MNVs did not exhibit cytotoxicity in kidney cells.

### [Example 3] Analysis of Delivery Ability (Mobility) within Cells/Organs

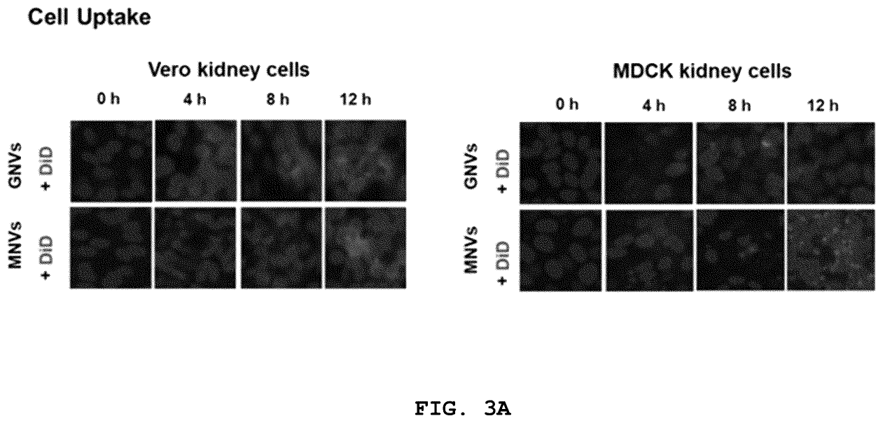

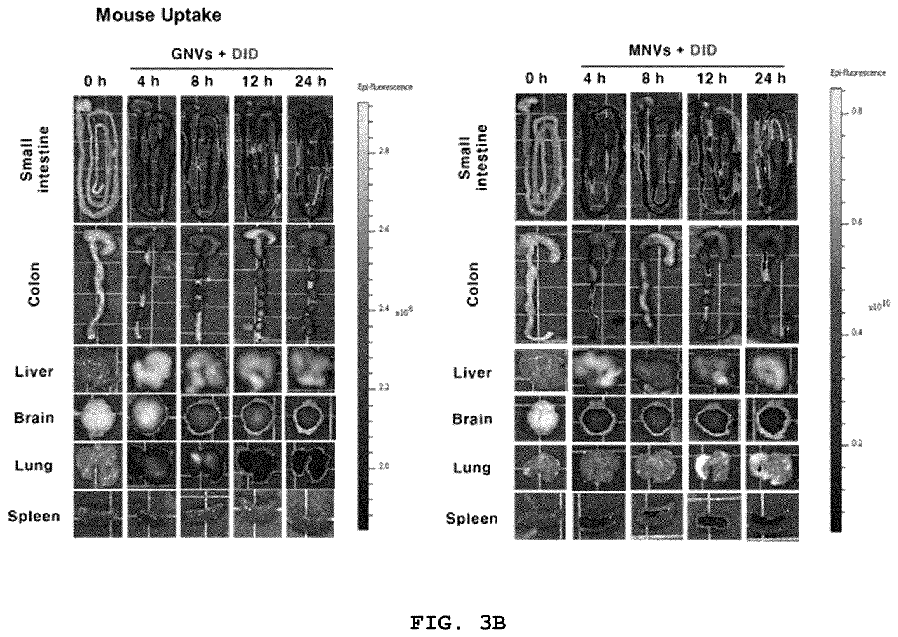

According to Experimental Example 4 above, as a result of analyzing whether the samples labeled with DiR was well absorbed in kidney cells/organs, as shown in FIGS. 3A and 3B, the samples were detected in kidney cells for 4 to 12 hours (FIG. 3A). In addition, signals generated by the samples appeared in the small intestine, large intestine, liver, and brain 4 hours after oral administration of the samples to mice, and only in the liver after 24 hours (FIG. 3B). From the results, it was confirmed that GNVs and MNVs labeled with DiR had excellent delivery ability (mobility) within cells/organs.

### [Example 4] Drug Delivery Ability Analysis

According to Experimental Examples 5 and 6 above, as a result of analyzing the drug delivery ability of the samples, as shown in FIGS. 4A to 4F, in the electroporation analysis (Experimental Example 5), it was confirmed that the uptake of mRNA-GFP loaded onto GNVs was more efficient compared to the control group (CON; mRNA-GFP not loaded onto GNVs) (FIG. 4B). In addition, the western blotting analysis (Experimental Example 6) showed that the expression level of HSP70 protein loaded onto GNVs was higher than that of HSP70 protein loaded onto MNVs (FIG. 4C). In addition, it was confirmed that the drug delivery efficiency of HBsAg protein loaded onto GNVs was better than that of MNVs (FIGS. 4D to 4F). From the results, it was confirmed that the drug (HBsAg) delivery ability of GNVs and MNVs was excellent, and among them, the drug (HBsAg) delivery ability of GNVs was more excellent.

### [Example 5] Immunogenicity Analysis of HBsAg

According to Experimental Examples 7 and 8 above, as a result of analyzing the effect of delivery of HBsAg through the samples on immunogenicity in animal models, as shown in FIGS. 5A and 5B, it was confirmed that 5 or 10 μg of HBsAg loaded onto the samples induced a higher level of antibody production compared to the control group (10 μg of HBsAg not loaded onto the samples) (FIG. 5B). From the result, it was confirmed that GNVs-mediated and MNVs-mediated delivery of HBsAg enhanced antibody response to the antigen.

As described above, specific parts of the present disclosure have been described in detail. For those skilled in the art, it is clear that these specific techniques are merely preferred examples and do not limit the scope of the present disclosure. That is, the practical scope of the present disclosure is defined by the appended claims and their equivalents.

What is claimed is:

1. A drug delivery composition comprising nano-vesicles derived from grapefruit (Citrus×paradisi) (hereinafter referred to as grapefruit derived nano-vesicles) or nano-vesicles derived from mandarin orange (Citrus reticulata) (hereinafter referred to as mandarin orange derived nano-vesicles) as an active ingredient.

2. The drug delivery composition of claim 1, wherein the grapefruit derived nano-vesicles have an average particle diameter in a range of 30 to 60 nm.

3. The drug delivery composition of claim 1, wherein the mandarin orange derived nano-vesicles have an average particle diameter in a range of 100 to 300 nm.

4. The drug delivery composition of claim 1, wherein the nano-vesicles improve delivery efficiency of a drug into cells.

5. The drug delivery composition of claim 1, wherein a drug is hepatitis B virus surface antigen (HBsAg) or influenza virus antigen.

6. A pharmaceutical composition for preventing or treating hepatitis B, the pharmaceutical composition comprising the nano-vesicles of claim 1, which are loaded with a hepatitis B treatment drug, as an active ingredient.

7. The pharmaceutical composition of claim 6, wherein a hepatitis B treatment drug is hepatitis B virus surface antigen (HBsAg).

8. A health functional food composition for preventing or alleviating hepatitis B, the health functional food composition comprising the nano-vesicles of claim 1, which are loaded with a hepatitis B treatment drug, as an active ingredient.
